(19) United States
(12) Patent Application Publication
(10) Pub. No.: US 2014/0079586 A1
(43) Pub. Date: Mar. 20, 2014

(54) Dispensing Device with Fan Directed Air Stream

(71) Applicant: The Schawbel Corporation, Bedford, MA (US)

(72) Inventors: Stephen Shapiro, Arlington, MA (US); Tom Paganetti, Bedford, MA (US)

(73) Assignee: The Schawbel Corporation, Bedford, MA (US)

(21) Appl. No.: 14/028,191

(22) Filed: Sep. 16, 2013

Related U.S. Application Data
(60) Provisional application No. 61/701,089, filed on Sep. 14, 2012.

(51) Int. Cl. A61L 9/03

(52) U.S. Cl. CPC A61L 9/032
USPC 422/4; 422/124

(57) ABSTRACT

A dispensing device for dispensing active ingredient such as insect repellant. The device. The device generates two distinct air streams. A first air stream contains the active ingredient and a second air stream generated by a mechanical blower assists in carrying the dispensed active ingredient.

### RELATED APPLICATIONS

This application claims the benefit of U.S. Prov. Appl. Ser. No. 61/701,089, filed on Sep. 14, 2012—the contents of which are incorporated by reference herein.

### FIELD OF THE INVENTION

The present invention relates generally to devices for dispensing volatile active substances. More particularly, the invention relates to a dispensing device that is provided with a blower for directing active ingredient to a desired area.

### BACKGROUND OF THE INVENTION

Handheld, portable devices that utilize heat to evaporate or volatilize active ingredients or other volatile substances are well known in the prior art. Such devices are commonly used to dispense insect control active ingredients or air treatment materials, such as air fresheners. Users employ these devices in a variety of places, such as in their backyards, near swimming pools or patios and the like.

Many dispensing devices utilize heat to volatize and dispense volatilizable substances. For instance, in some devices a conductive heat plate is provided—upon which a mat impregnated with a volatilizable substance is placed. When the heat plate is sufficiently heated—the volatilizable substances evaporate and are thereby dispensed.

In such systems, the heat source causes a rising hot air current that is directed upwardly. Repellant or other substances to be dispensed are carried upward in the rising hot air stream. However, this system may be suboptimal for several reasons. First, the repellant devices are often placed off to the side of a site whereby repellant is desired. For instance, picnickers, gardeners, or hunters often place the dispensing device in an area near where they are situated. Because the device creates an upward stream—repellant or other active ingredient is not directed to where it is needed most.

Moreover, some active ingredients carried in the hot air stream are rather heavy as compared to pure air. As a result, it may be difficult to achieve wide dispersal of active ingredient relying on a hot air stream alone.

There is therefore a need in the art for a dispensing device that is capable of directing an air stream containing active ingredient to an area of interest. Moreover, there is a need for dispensing device whereby active ingredient particles are propelled beyond the confines of a hot air stream.

### SUMMARY OF THE INVENTION

The current invention addresses these problems by providing a dispensing device that contains an adjustable blower, such as a fan, for blowing airborne active ingredient. The fan directs the air containing active ingredient toward a desired area and it also propels active ingredient particles to travel distances greater than they would normally travel unassisted by the fan.

The air stream must entrain the particles or vapor of active ingredient after they have been volatilized by heating. It is desirable that the air circulation created by the fan not interfere with the vaporization caused by heating the active ingredient. The inventive system achieves that end with a two stage process whereby, first the active ingredient is incorporated into the air by heating and then secondarily, the air/active ingredient mixture is energized by a mechanical fan or other blowing device to flow further from the dispenser than it would without such a fan. The fan or air blowing device may be mounted in such a way as to allow the user to direct the flow of active ingredient in a more favorable direction than without the fan.

### BRIEF DESCRIPTION OF THE DRAWINGS

FIG. 1 is a left, perspective view of a dispensing unit and an attached blower unit according to an embodiment of the invention.

FIG. 2 is a right, perspective view of a dispensing unit and an attached blower unit according to an embodiment of the invention.

FIG. 3 is a front, cross-sectional view of a dispensing unit and an attached blower unit according to an embodiment of the invention.

FIG. 4 is a bottom view of a dispensing unit and an attached blower unit according to an embodiment of the invention.

### DETAILED DESCRIPTION OF THE INVENTION

Embodiments of the present invention will now be described with reference to the above-identified Drawings. However, the Drawings and the description herein of the invention are not intended to limit the scope of the invention. It will be understood that various modifications of the present description of the invention are possible without departing from the spirit of the invention, Also, features described herein may be omitted, additional features may be included, and/or features described herein may be combined in a manner different from the specific combinations recited herein, all without departing from the spirit of the invention.

In the inventive device, active ingredient is volatized and released into the air. In embodiments of the invention, a hydrocarbon fuel source is utilized to sustain a flame for dispensing the volatilizable substances. For example, the dispenser may be similar to the one disclosed in any of U.S. Pat. No. 5,700,430 or U.S. Pat. No. 5,928,605—each of which is incorporated by reference herein. The device, in principle, includes a burner that is capable of sustaining flame, a catalytic structure, a candle, or an electric heating element to heat and volatize active ingredient. In the embodiment described herein, a combustion nozzle located in a combustion chamber is used as a heat source to volatize active ingredients. The combustion nozzle supports a flame produced by hydrocarbon combustion, which heats a heating plate positioned above the combustion chamber. The heating plate is made of thermally conductive material—such as aluminum, copper or the like. In use a pad or mat that is impregnated with a volatilizable substance is placed on the heating plate. When a flame is ignited (or a heater is initiated), the heating plate reaches sufficient temperature to cause evaporation of the volatilizable substance. The evaporated substance is then introduced into the ambient air.

The active ingredient that is introduced into the ambient air in this manner is not directed to a specific region and because the active ingredient is heavier than air—it may not travel a far enough distance for maximal efficacy. The inventive device provides an air stream to assist in carrying at least a portion of active ingredient to distances it would not otherwise travel and it allows for more focused delivery of active ingredient. In an embodiment of the invention, active ingredient to be dispensed is insect repellant; however, it will be understood that the dispersal of any of different active ingredients are within the teaching of the invention.

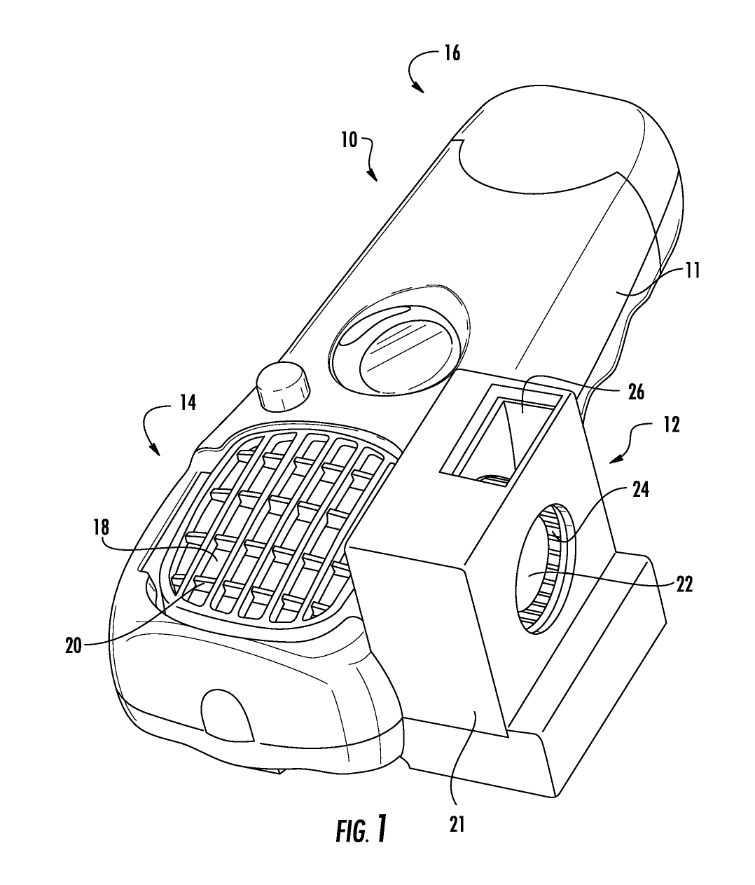

FIG. 1, shows a dispenser unit 10 having a blower unit 12 associated therewith. As shown, the dispenser unit 10 has an outer housing 11 or casing that holds the internal mechanical components of the system. The housing has a dispensing end 14 and an elongated handling end 16. The dispensing end 14 is comprised of a heating plate 18 upon which a mat (not shown) is placed. A protective grill 20 is attached above and substantially parallel to the heating plate 18.

A blower unit 12 is shown connected to the outer housing 11 at the dispensing end. The blower unit has an outer housing 21, which encases a chamber having a fan, pump, impeller, vacuum or similar blowing apparatus located therein. The outer housing as an opening 22 or similar aperture or channel allowing for the intake of air. Air enters through opening 22 and it is directed upward by an internal fan 24 and then out of the chamber through a second opening or vent 26. An air stream emanates (upwardly in the embodiment shown) from the vent 26 as the fan continuously draws in air and redirects it to be forced out of vent 26.

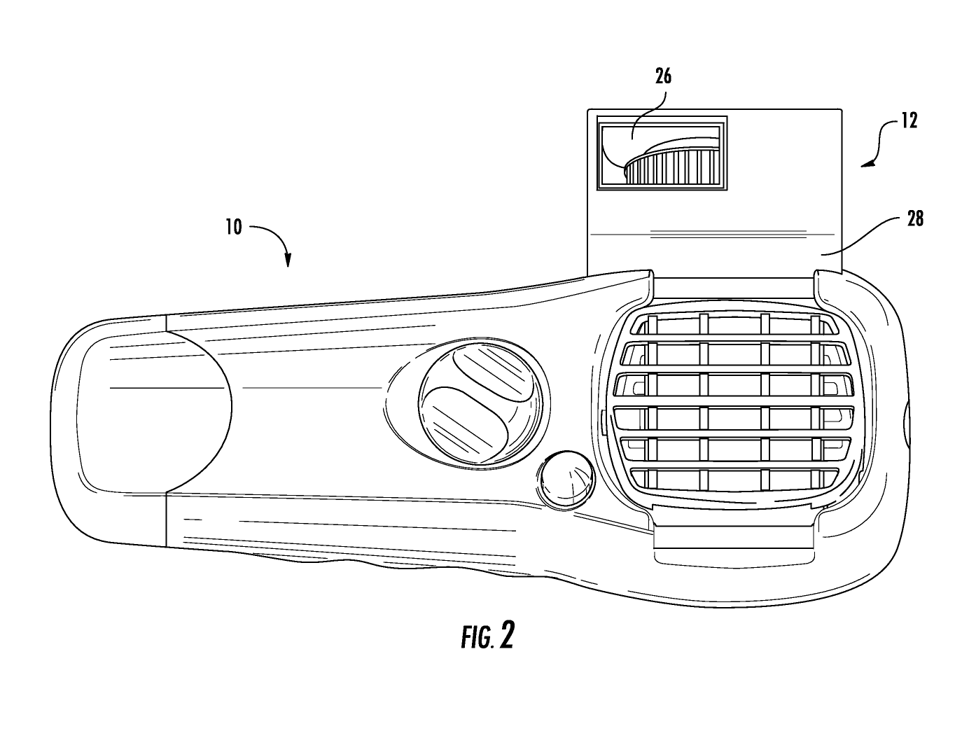

FIG. 2 shows a view of the dispenser unit which shows a top/rear view of the blower unit 12. As shown, the rear wall 28 of blowing unit 12 is a solid wall and it does not have any air intake openings. This is done to reduce any likelihood that air drafts caused by directed movement of air into the blower unit would interfere with vaporization and/or disturb the temperature of the heating plate 18. In this respect, in a preferred embodiment of the invention, an air intake aperture associated with a blower unit that is used to create an air stream draws air from an area that is far enough from dispensing activities so as not to interfere with the same. In the embodiment shown in the Figures, a wall 28 serves as a partial barrier separating the dispensing air stream and the air intake opening 22. However, it will be understood that in other embodiments, a physical barrier is not necessary and the air intake may be situated in a manner such that it does not face the heating plate and the air stream formed thereby. Alternatively, the air intake aperture may be positioned at a sufficient distance so that it does not interfere with the evaporation on the heating and it is effectively separated by space.

When the dispenser 10 is engaged, active ingredient rises into the air above the heating plate. When the blowing unit 12 is simultaneously engaged, the resulting air stream propels particles of active ingredient to distances they would have otherwise traveled unassisted by a generated air stream.

In one embodiment of the invention, a battery operated fan is provided, but it will be understood that a fan or similar blowing apparatus may be powered by way of a line cord to a power outlet.

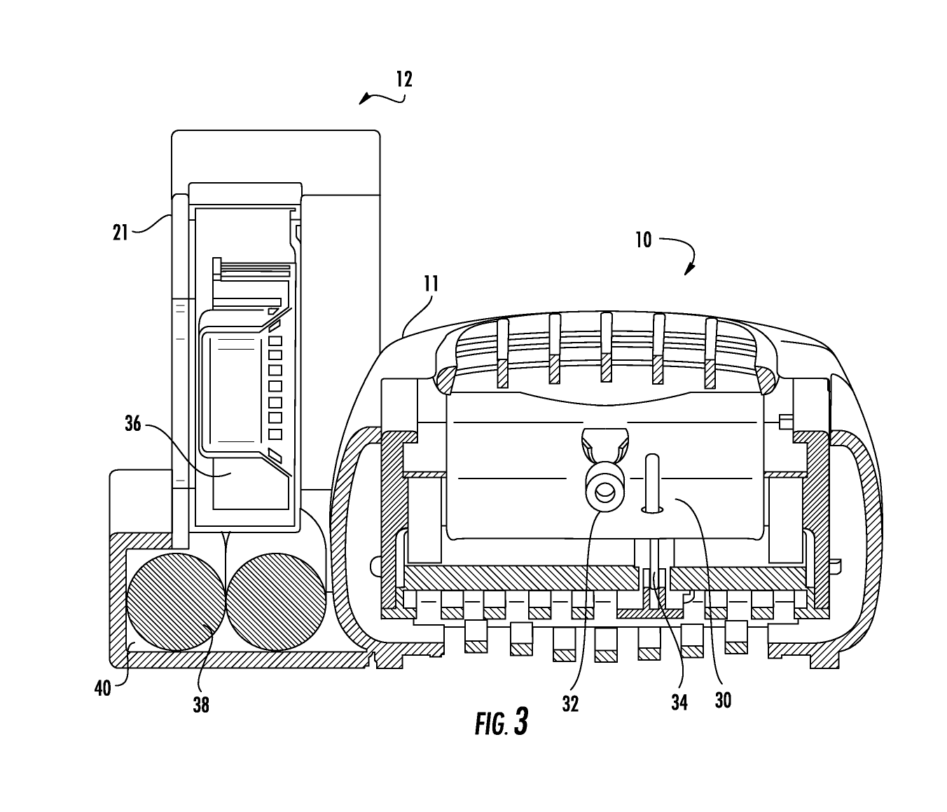

FIG. 3 shows a cross-sectional view through front end of a dispenser unit 10 and attached blower unit 12. Outer casing 11 is shown housing a combustion chamber 30, which includes a combustion nozzle 32 and an air intake channel 34 to deliver air necessary to support hydrocarbon combustion. A heating plate 18 is shown disposed above the combustion chamber 30. Blower unit 12 is shown attached to the sidewall of the dispensing end 14 of the unit. As shown, outer casing 21 houses a chamber 36 which contains a fan 24 or similar blowing apparatus. In the embodiment shown, the fan 24 is powered by batteries 38, which are shown located in a battery box 40 beneath the fan chamber 36.

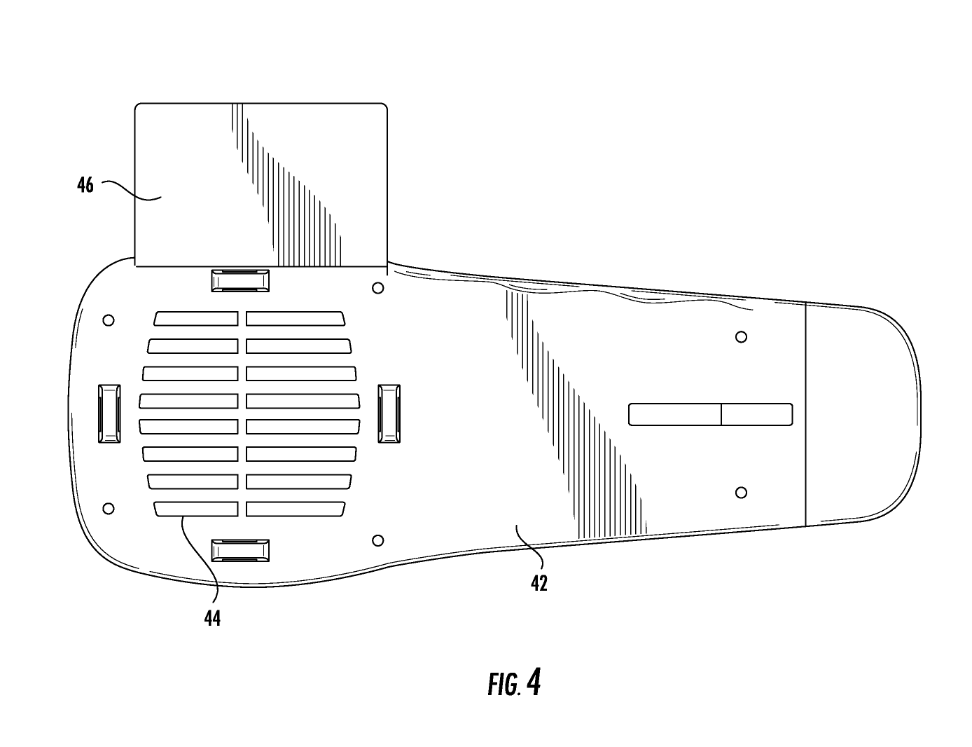

FIG. 4 shows a bottom surface 42 of the dispenser housing showing air vents 44 for the heating system and the bottom outer surface 46 of battery box 40. The bottom surface 42, 46 rests on a flat surface during operation of the unit.

It will be understood that the blower unit 12 need not be permanently mounted to the dispenser unit and that in embodiments of the invention, the blower unit 12 may be an independent unit that is selectively detachable from the dispensing unit 10. It will be understood that the blower unit 12 may be temporarily attached to the dispensing unit 10 by way of snaps, Velcro®, screws, hooks or any of various male-female connection mechanisms. Moreover, the invention is not limited to a single blower unit and more than one blower unit may be mounted (either permanently or temporarily) to the dispenser unit. In one embodiment, a second blower unit (not shown) may be attached to the side wall of the dispensing unit on the opposite side of the blower unit shown in the Figures herein.

In an embodiment of the invention, the blower unit 12, is pivotably mounted to the device in a manner in which it may be rotated by as much as 360°. That is, the blower unit 12 may be mounted by way of a pivot which allows incremental movement of the blower unit 12 thereabout.

It will be understood that the invention set forth herein, broadly relates to an apparatus and method of creating two independent air streams. A first air stream is generated by rising heat causing volitilzable substances to vaporize and enter into the air. A second, independent air stream is generated by a mechanical blowing device. The air stream generated by the blowing device energizes the particles of active ingredient generated in the first air stream and propels them to distances that are greater than the particles would have otherwise traveled unassisted by a secondary air stream. Preferably, the first air stream and second air stream are generated in a side-by-side arrangement. Namely, a hot air stream is positioned adjacent to an air stream generated by a mechanical blower.

In one embodiment of the invention, directable louvers are provided on vent 26 to direct an air stream emanating therefrom. A motor control also can be incorporated to control air flow rate. In this manner, the direction and rate of the flow of active ingredient may be easily selected and adjusted by rotating the blower unit and/or louvers and to a desired position and by adjusting the motor to control flow rate.

Embodiments of the invention, thus allows for a more tailored experience where active ingredient is directed to an area where it is needed most. Moreover, the active ingredient is carried further and it covers greater area than it would otherwise travel unassisted by a fan.

Having described this invention with regard to specific embodiments, it is to be understood that the description is not meant as a limitation since further modifications and variations may be apparent or may suggest themselves to those skilled in the art. It is intended that the present application cover all such modifications and variations.

What is claimed is:

1. A dispenser apparatus, comprising;
a heat source for heating a volitilizable substance;
a heat plate for receiving a mat impregnated with volitilzable substance disposed above of said heat source;
said heat source and said heat plate generating a first air stream of rising hot air,
said hot air comprising particles of said volitizable substance;
an air blower, said air blower being positioned substantially adjacent to said heating plate;
said air blower generating a second air stream;
whereby said second air stream generated by said air blower carries particles of said volitizable substance.

2. The dispenser apparatus of claim 1, whereby said heat source comprises a flame supported by hydrocarbon combustion.

3. The dispenser apparatus of claim 1, whereby said blower comprises an electric fan.

4. The dispenser apparatus of claim 3, whereby said fan is located in a chamber.

5. The dispenser apparatus of claim 4, whereby said chamber comprises an air intake opening and a vent for allowing air to exit from said chamber.

6. The dispenser apparatus of claim 4, whereby said air intake opening does not face said first air stream.

7. The dispenser apparatus of claim 1, whereby said air blower comprises a unit having an outer casing, said outer casing enclosing a fan, said outer casing having a an air intake opening and a vent, said unit being removable from said dispenser apparatus.

8. The dispenser apparatus of claim 1, whereby said air blower is mounted to a sidewall of said dispenser apparatus.

9. The dispenser apparatus of claim 1, whereby said blower air blower is movable, whereby when said air blower is moved, said second air stream is moved.

10. A method of dispensing an active ingredient, comprising the steps of:
placing a mat impregnated with said active ingredient in thermal communication with a heat source;
activating said heat source;
generating a first air stream, said first air stream comprising rising hot air and particles of said active ingredient;
generating a second air stream substantially adjacent to said first air stream using a mechanical air blowing apparatus;
whereby said second air stream caries a portion of said active ingredient from said first air stream.

11. The method of claim 10, further comprising the step of moving said mechanical blowing apparatus to change a direction of said second air stream.

12. The method of claim 10, further comprising the step of moving louvers on said mechanical blowing apparatus to change a direction of said second air stream.

13. The method of claim 10, further comprising the step of intaking air into said blowing apparatus and releasing air from said blowing apparatus.

14. The method of claim 13, whereby said step of intaking air comprises the step of drawing air from an area that is not facing said first air stream.
